(19) United States
(12) Patent Application Publication
(10) Pub. No.: US 2023/0279329 A1
(43) Pub. Date: Sep. 7, 2023

(54) IN-SITU SERVO-HYDRAULIC BIO-MANIPULATOR

(71) Applicant: University of Florida Research Foundation, Inc., Gainesville, FL (US)

(72) Inventors: Jack E. FAMIGLIETTI, Sarasota, FL (US); Sean R. NIEMI, Gainesville, FL (US); Wallace G. SAWYER, Gainesville, FL (US); Ryan A. SMOLCHEK, Gainesville, FL (US); Kylie E. VAN METER, Tallahassee, FL (US)

(21) Appl. No.: 18/013,359

(22) PCT Filed: Jul. 1, 2021
(86) PCT No.: PCT/US2021/040073

§ 371 Date: Dec. 28, 2022

Related U.S. Application Data
(60) Provisional application No. 63/046,971, filed on Jul. 1, 2020.

(51) Int. Cl. C12M 1/26; G02B 21/32; C12M 1/00; C12M 3/00; C12M 1/12; C12M 1/34; B25J 7/00

(52) U.S. Cl. CPC C12M 33/04; B25J 7/00; C12M 21/08; C12M 25/04; C12M 29/10; C12M 41/36; G02B 21/32

(57) ABSTRACT

Disclosed herein are systems and methods relating to in-situ servo-hydraulic biomanipulators. In-situ servo-hydraulic bio-manipulators as described herein have the advantages over existing systems and methods at least by having a lower cost, interchangeable and/or disposal displacement devices coupled to the extrusion head, and mounting of the displacement head along the optical axis of a microscope for enhanced visibility and well clearance.

### CROSS REFERENCE TO RELATED APPLICATION[S]

This application claims the benefit of and priority to U.S. Provisional Pat. Application No. 63/046,971 entitled “IN-SITU SERVO-HYDRAULIC BIO-MANIPULATOR,” filed on Jul. 1, 2020, which is expressly incorporated by reference as if fully set forth herein in its entirety.

### BACKGROUND

Culturing cells in a 3D environment yields cellular behavior and morphology that more closely matches what is observed in the human body. 3D microgels used for this kind of culturing have a unique attribute: cells can be deposited in specific locations in 3D space and remain in position for extended time periods. This enables the creation of complex structures and co-culture environments where cellular interactions and developments over time are observed.

Such advantages of 3D culture are not without their challenges, however. The level of precision necessary to perform this placement is nearly impossible by hand, and without a visually guided system or near perfect metrology, it is very difficult to place or sample material exactly where needed. Furthermore, existing micro-manipulators are oriented at an angle mounted away from the optical axis, reducing clearance and limiting area of a well in which a cell or cells can be placed/retrieved.

Accordingly, there is a need to address the aforementioned deficiencies and inadequacies.

### SUMMARY

Disclosed herein are systems and methods relating to in-situ servo-hydraulic bio-manipulators.

In embodiments, an in-situ servo-hydraulic bio-manipulator, can comprise: a micro-displacement hydraulic controller; a macro-displacement hydraulic controller; a junction box, wherein a portion of the junction box is optically transparent; an extrusion head in fluidic communication with the junction box, micro-displacement controller, and macro-hydraulic controller; and an adapter configured to mechanically couple the extrusion head to the optical axis of a microscope.

In embodiments, the extrusion head can further comprise an adapter configured to receive interchangeable tips.

In embodiments, the adapter configured to receive interchangeable tips is a tapered nozzle configured to receive micropipette tips, a tapered nozzle with an orifice configured to receive a glass capillary, or a sealed adapter configured to receive a luer-lock syringe needle.

In embodiments, the micro-displacement hydraulic controller comprises an internal sealing assembly.

In embodiments, the micro-displacement hydraulic controller and macro-displacement hydraulic controller are in fluidic communication with the junction box through tubing filled with a first fluid, and the extrusion head and junction box are in fluidic communication though tubing filled with a second fluid, wherein the second fluid is different than the first fluid.

In embodiments, first fluid is a non-biocompatible fluid. In embodiments, the first fluid is non-compressible. In embodiments, the first fluid is Novec 7500.

In embodiments, the second fluid forms an immiscible layer with the first fluid in the junction box. In embodiments, the second fluid is a bio-compatible fluid. In an embodiment, the second fluid is phosphate-buffered saline (PBS).

In embodiments, the micro-displacement hydraulic controller and macro-displacement hydraulic controller each comprise a threaded shaft, the threaded shaft of the macro-displacement hydraulic controller being larger in diameter than the threaded shaft of the micro-displacement hydraulic controller.

In embodiments, the threaded shaft of the of the macro-displacement hydraulic controller is a ½-13 UNC threaded shaft. In embodiments, the threaded shaft of the of the macro-displacement hydraulic controller is a 3/16-100 UNUF threaded rod.

In embodiments, the micro-displacement hydraulic controller and macro-displacement hydraulic controller each comprise a dial capable of being operated independently of the other.

Further described herein are a bio-manipulation systems. In embodiments, systems as described herein can comprise: an in-situ servo-hydraulic bio-manipulator as described herein; and a bioreactor. In embodiments, the bioreactor is a perfusion-enabled bioreactor. In embodiments, the perfusion-enable bioreactor comprises a passive negative constant pressure device.

Systems as described herein further comprise a 3D cell growth media in the bioreactor. In embodiments, the 3D cell growth media is a Herschel-Bulkley fluid having a yield stress of less 100 pascals.

Described herein are methods of using an in-situ servo-hydraulic bio-manipulator, comprising: providing an in-situ servo-hydraulic bio-manipulator as described herein; providing one or more mammalian cells; and translating the position of the one or more mammalian cells by operating the micro-displacement hydraulic controller, macro-displacement hydraulic controller, or both.

In further aspects, described herein are methods of using an in-situ servo-hydraulic bio-manipulator, comprising: providing an in-situ servo-hydraulic bio-manipulator as described herein; providing one or more inorganic signaling markers; and translating the position of the one or more inorganic signaling markers by operating the micro-displacement hydraulic controller, macro-displacement hydraulic controller, or both.

The details of one or more embodiments of the present disclosure are set forth in the accompanying drawings and the description below. Other features, objects, and advantages of the present disclosure will be apparent from the description and drawings, and from the claims.

### DESCRIPTION OF DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A-1B depict a schematic showing an embodiment of an in-situ servo-hydraulic bio-manipulator as described herein as well as a micrograph of visual feedback of the tip and manipulated material with such (FIG. 1B).

FIG. 2 is an embodiment of a confocal compatible incubator which can be used as part of a bio-manipulation system in conjunction with in-situ servo-hydraulic bio-manipulators as described herein.

FIG. 3 is an embodiment of a perfusion-enabled bioreactor which can be used as part of a bio-manipulation system in conjunction with in-situ servo-hydraulic bio-manipulators as described herein. The embodiment of FIG. 3 can be utilized along with negative pressure vessels to enable perfusion of fluids (delivery of nutrients and removal of cellular waste) throughout the bioreactor.

FIG. 4 is an embodiment of an application of in-situ servo-hydraulic bio-manipulators (and systems comprising such) according to the present disclosure.

FIG. 5 is a confocal fluorescent micrograph showing additional aspects related to FIG. 4.

FIGS. 6A-6C show an embodiment of a macro-displacement hydraulic controller according to the present disclosure.

FIGS. 7A-7C show an embodiment of a micro-displacement hydraulic controller according to the present disclosure.

FIGS. 8A-8C show an embodiment of an internal sealing assembly for a micro-displacement hydraulic controller according to the present disclosure.

FIG. 9 is a photograph showing a reduced-to-practice embodiment of a bio-manipulator coupled a confocal microscope. Bio-manipulator controls and assemblies shown in a standard working layout. The dial assemblies are located near the operator for making small volume displacements. During a print, the turret head assembly is tilted over the culture infrastructure and the needle tip is lowered into the sample using the geared travel native to the confocal microscope. The material manipulation is completed using either brightfield or fluorescent imaging, after which the needle is raised out of the sample and tilted away before removal.

FIG. 10 is a photograph showing a reduced-to-practice embodiment of a bio-manipulator controller assembly of a bio-manipulator as described herein. The controller assembly shown with both the coarse (150 uL/revolution) and fine (0.5 uL/revolution) dial assemblies integrated into the Delrin/stainless steel frame. Rotation of either dial produces axial travel along its respective internal thread helix, which in turn displaces the Novec 7500 engineering fluid. The coarse dial incorporates a 1 /2-13 UNC thread, while the fine dial incorporates a 3/16-100 UNEF thread. Both dials can be operated independently to accomplish certain tasks, with the fine dial being used for manipulating biomaterials while the coarse dial is used for both clearing needle tips and exchanging turret assemblies. Dial assemblies are connected by a T-fitting which is then connected to the transparent junction box through flexible tubing.

FIG. 11 is a photograph showing a reduced-to-practice embodiment of a bio-turret head assembly for a bio-manipulator as described herein. Turret head assembly shown with the syringe needle attachment variant. The acrylic junction box (shown affixed to the column of the confocal microscope) provides visibility to the Novec 7500 engineering fluid and Phosphate Buffered Saline (PBS) immiscible layer. The location of the transparent junction box can be adjusted to reduce the height of the column of liquid acting at the printing interface to prevent unintended flow/suction. Different printing heads can be attached to the junction box through removal of the flexible tubing with care not to introduce any unintended cavities or bubbles.

FIG. 12 is a photograph showing a reduced-to-practice embodiment of a bio-turret head assembly for a bio-manipulator as described herein. Illustration of a typical print/extraction setup using the syringe needle variant of the turret head assembly. The turret head assembly mounts to the turret of the confocal microscope and is aligned along the optical axis simplifying the process of locating the needle tip relative to the materials being manipulated. In addition, the vertical orientation of the needle and turret head assembly improves upon the versatility of the system when working with culture plates and infrastructure with relatively tall cavities. Traditional micro-manipulators are oriented at an angle mounted away from the optical axis, reducing clearance. The stage of the confocal microscope translates on the x-y coordinate plane, while the turret is translated along the z-axis using a geared-head. When not in use, the turret head assembly is tilted away from the print-site and disconnected.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

About: The term “about”, when used herein in reference to a value, refers to a value that is similar, in context to the referenced value. In general, those skilled in the art, familiar with the context, will appreciate the relevant degree of variance encompassed by “about” in that context. In an embodiment, “about” means a range encompassing +/- 10% of the reference value. In an embodiment, “about” means a range encompassing +/- 5% of the reference value.

Associated with: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition”, as used herein, can be used to refer to a discrete physical entity that comprises one or more specified components. In general, unless otherwise specified, a composition can be of any form - e.g., gas, gel, liquid, solid, etc.

Comprising: A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

“Improved,” “increased” or “reduced”: As used herein, these terms, or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: as used herein, “sample” refers to one or more biological substances (preferable a mammalian cell or plurality of mammalian cells) whose position can be translated using systems and methods as described herein.

### Discussion

Described herein are systems and methods related to in-situ servo-hydraulic bio-manipulators. The use of an in situ hydraulic actuator attached directly to a microscope allows for precise control of locational placement, sampling of biological materials, and the ability to observe long term cellular interactions without disturbing an experiment. Such systems and methods allow for quick and simple retrieval of deposited biological materials as well.

In embodiments as described below, the actuator can use two piston-style hydraulic pumps to control course and fine displacement of volumes. The piston dimensions can be tuned to provide a range of precision and pumping speed. Pistons can be driven by lead-screws and can be actuated manually or via electric motors. The motors and piston-pumps can be located remotely to remove vibrations from the imaging setup. The use of hydraulics can allow for smooth motion while damping out vibrations from motors or other external sources, preventing them from affecting the desired structure or imaging quality. The extrusion head can be fixed to an adapter that allows mounting to the condenser head of an inverted microscope, aligning the manipulator with the optical axis of imaging equipment. The extruding head is designed to be compatible with a variety of common cell culture instruments (pipette tips, needles, capillary tubes, etc.)

### In-situ Servo-Hydraulic Bio-Manipulators

Described herein are systems and methods relating to in-situ servo-hydraulic bio-manipulators. In embodiments as described herein, in-situ servo-hydraulic bio-manipulators comprise: a micro-displacement hydraulic controller; a macro-displacement hydraulic controller; a junction box, wherein a portion of the junction box is optically transparent; an extrusion head in fluidic communication with the junction box, micro-displacement controller, and macro-hydraulic controller; and an adapter configured to mechanically couple the extrusion head to the optical axis of a microscope.

Systems and micromanipulators as described herein can comprise a micro-displacement hydraulic controller. The micro-displacement hydraulic controller can comprise an internal sealing assembly, a piston-style actuator (such as a threaded rod), and a fluid connector to connect the controller to a fluid line. In an embodiment, the piston-style actuator of the micro-displacement hydraulic controller can comprise a 3/16-100 UNUF threaded rod. The piston-style actuator of the micro-displacement hydraulic controller can have a shaft with a threaded portion and a non-threaded portion, the threaded portion having a larger diameter than the non-threaded portion. Micro-displacement hydraulic controllers can also comprise a dial operably connected to the piston-style actuator (and having a larger diameter than the actuator) exterior to the housing that the user can utilize to actuate the fluid.

Systems and micromanipulators as described herein can comprise a macro-displacement hydraulic controller. The macro-displacement hydraulic controller can comprise a housing, an internal sealing assembly, a piston-style actuator, gaskets to prevent fluid leakage, and screws to hold the assembled controller together. In an embodiment, the piston-style actuator of the macro-displacement hydraulic controller can comprise a ½-13 UNC threaded shaft. The piston-style actuator can have a shaft with a threaded and non-threaded portion. The threaded portion of the shaft of the macro-displacement hydraulic controller can have a diameter larger than the shaft (or rod) itself of the micro-displacement hydraulic controller. Macro-displacement hydraulic controllers can also comprise a dial operably connected to the piston-style actuator (and having a larger diameter than the actuator) exterior to the housing that the user can utilize to actuate the fluid.

The micro-displacement hydraulic controller and macro-displacement hydraulic controller each comprise a dial capable of being operated independently of the other.

The extrusion head can be mounted along the optical axis of a microscope (for example a confocal microscope) with the use of an adapter that can mechanically couple the extrusion head to the microscope (in particular the optical turret of a microscope, such as the optical turret of a Nikon A1R confocal microscope).

The extrusion head can further be configured for interchangeable or otherwise disposal extrusion devices (also referred to herein as “tips” or “displacement tips”). The extrusion head can be configured to receive interchangeable tips by way of a tapered nozzle configured to receive commercially available plastic micropipette tips (for example 10, 20, 200, 1000 µL tips. In embodiment, the extrusion head can comprise a tapered nozzle with an orifice configured to receive a glass capillary. In an embodiment, the extrusion head can comprise a sealed adapter configured to receive a luer-lock syringe needle. In further embodiments, bio-manipulators as described herein can further comprise displacement tips that can interact with (manipulate, place, etc.) biological material. Such displacement tips can be detachably connected through an adapter operably connected with the extrusion head (for example through an interference fit or threaded screw fit). In other embodiments, the displacement tips may be fixed to the extrusion head in a non-detachable manner. In an embodiment, the tip can be a tapered nozzle configured to interface with micropipette displacement tips (for example 10, 20, 200, 1000 microliter disposable micropipette tips, sterilized or not) through an interference fit. In other embodiments, the tip can be configured to hold a glass capillary for the manipulation of single cells and ultra-low volume dispersions. In other embodiments, the tip can be a sealed adapter compatible with syringe needles, for example Luer-Lock syringe needles.

The micro-displacement hydraulic controller and macro-displacement hydraulic controller can be in fluidic communication with the junction box through tubing filled with a first fluid, and the extrusion head and junction box are in fluidic communication though tubing filled with a second fluid, wherein the second fluid is different than the first fluid. The junction box can comprise an optically transparent portion that allows for observation of the immiscible layer of the first and second fluid. In embodiments, the optically transparent portion can be constructed of clear acrylic such as those known in the art.

The first fluid can a non-biocompatible fluid. The first fluid can prevent the formation of bubbles. The first fluid can be Novec 7500. Novec 7500 engineered fluid was selected for the present system for its inherent low kinematic viscosity and low propensity of trapping bubbles which would introduce compressibility. Novec 7500 is also compatible with the elastomers used for sealing, is environmentally friendly, and immiscible with PBS (an embodiment of a biocompatible buffer fluid). This fluid was designed as an alternative to perfluorocarbons (PFCs) and perfluoropolyethers (PFPEs) by 3 M to reduce the presence of high Global Warming Potential (GWP) and flammable liquids in semiconductor systems. Alternatives to Novec 7500 according to the present disclosure include any low-viscosity alternative, so long as compressibility and immiscibility with the biocompatible buffer fluid are maintained.

The second fluid can form an immiscible layer with the first fluid in the junction box. The second fluid is a bio-compatible fluid. The second fluid can be phosphate-buffered saline (PBS). Phosphate Buffered Saline (PBS) is a widely used buffer solution used in cell culture and is present in an embodiment of the present system to prevent the first fluid (the non-compressible fluid such as Novec 7500 engineered fluid) from contacting sensitive biological materials and tissues. PBS can be substituted for any alternative buffer solution so long as the selected solution is biocompatible with the materials being manipulated and immiscible with the engineered fluid.

Also described herein are methods of using an in-situ servo-hydraulic bio-manipulator as described herein. Methods of using an in-situ servo-hydraulic bio-manipulator can comprise providing an in-situ servo-hydraulic bio-manipulator as described herein; providing one or more mammalian cells; and translating the position one or more mammalian cells by operating the micro-displacement hydraulic controller, macro-displacement hydraulic controller, or both.

### Systems

In-situ servo-hydraulic bio-manipulators as described herein can be part of systems for the placement, growth, and retrieval of biological materials, for example cells or spheroids comprised of a plurality of cells. Additional aspects of systems as described herein include 3D medium (also referred to herein as 3D culture medium or 3D cell culture medium), which comprises a plurality of packed hydrogels forming a granular, liquid-like solid. Such hydrogels can be swollen with a liquid, for example cell culture medium. In certain aspects, 3D culture medium is a Herschel-Bulkley fluid having a yield stress of less than 100 pascals to avoid the formation of crevasses and to provide cells an environment in which they are not too constrained (as to prevent efficient nutrient deliver, waste removal, cellular migration and/or expansion).

Systems as described herein can also further comprise one or more bioreactors, details of which can be found below. In certain aspects, bioreactors according to the present disclosure can be perfusion-enabled bioreactors. In certain aspects, bioreactors according to the present disclosure can be perfusion-enabled with a constant negative pressure device as described herein.

### 3D Cell Growth Medium

In certain aspects, bio-manipulators as described herein can be used in conjunction with biological samples and liquid-like solid (LLS) three-dimensional (3D) cell growth medium, as further described below.

Liquid-like solid (LLS) three-dimensional (3D) cell growth medium for use in with the disclosed bio-manipulator system is disclosed in WO2016182969A1 by Sawyer et al., which is incorporated by reference in its entirety for the description of how to make and uses this LLS medium.

Briefly, the 3D cell growth medium may comprise hydrogel particles dispersed in a liquid cell growth medium. Any suitable liquid cell growth medium may be used; a particular liquid cell growth medium may be chosen depending on the types of cells which are to be placed within the 3D cell growth medium. For example, suitable cell growth medium may be human cell growth medium, murine cell growth medium, bovine cell growth medium or any other suitable cell growth medium. Depending on the particular embodiment, hydrogel particles and liquid cell growth medium may be combined in any suitable combination. For example, in some embodiments, a 3D cell growth medium comprises approximately 0.5% to 1% hydrogel particles by weight.

In accordance with some embodiments, the hydrogel particles may be made from a bio-compatible polymer.

The hydrogel particles may swell with the liquid growth medium to form a granular gel material. Depending on the particular embodiment, the swollen hydrogel particles may have a characteristic size at the micron or submicron scales. For example, in some embodiments, the swollen hydrogel particles may have a size between about 0.1 µm and 100 µm. Furthermore, a 3D cell growth medium may have any suitable combination of mechanical properties, and in some embodiments, the mechanical properties may be tuned via the relative concentration of hydrogel particles and liquid cell growth medium. For example, a higher concentration of hydrogel particles may result in a 3D growth medium having a higher elastic modulus and/or a higher yield stress.

According to some embodiments, the 3D cell growth medium may be made from materials such that the granular gel material undergoes a temporary phase change due to an applied stress (e.g. a thixotropic or “yield stress” material). Such materials may be solids or in some other phase in which they retain their shape under applied stresses at levels below their yield stress. At applied stresses exceeding the yield stress, these materials may become fluids or in some other more malleable phase in which they may alter their shape. When the applied stress is removed, yield stress materials may become solid again. Stress may be applied to such materials in any suitable way. For example, energy may be added to such materials to create a phase change. The energy may be in any suitable form, including mechanical, electrical, radiant, or photonic, etc.

Regardless of how cells are placed in the medium, the yield stress of the yield stress material may be large enough to prevent yielding due to gravitational and/or diffusional forces exerted by the cells such that the position of the cells within the 3D growth medium may remain substantially constant over time. As described in more detail below, placement and/or retrieval of groups of cells may be done manually or automatically.

A yield stress material as described herein may have any suitable mechanical properties. For example, in some embodiments, a yield stress material may have an elastic modulus between approximately 1 Pa and 1000 Pa when in a solid phase or other phase in which the material retains its shape under applied stresses at levels below the yield stress. In some embodiments, the yield stress required to transform a yield stress material to a fluid-like phase may be between approximately 1 Pa and 1000 Pa. In some embodiments, the yield stress may be on the order of 10 Pa, such as 10 Pa +/-25%. When transformed to a fluid-like phase, a yield stress material may have a viscosity between approximately 1 Pa s and 10,000 Pa s. However, it should be understood that other values for the elastic modulus, yield stress, and/or viscosity of a yield stress material are also possible, as the present disclosure is not so limited.

A group of cells may be placed in a 3D growth medium made from a yield stress material via any suitable method. For example, in some embodiments, cells may be injected or otherwise placed at a particular location within the 3D growth medium with a syringe, pipette, or other suitable placement or injection device. In some embodiments an array of automated cell dispensers may be used to inject multiple cell samples into a container of 3-D growth medium. Movement of the tip of a placement device through the 3D growth medium may impart a sufficient amount of energy into a region around the tip to cause yielding such that the placement tool may be easily moved to any location within the 3D growth medium. In some instances, a pressure applied by a placement tool to deposit a group of cells within the 3D growth medium may also be sufficient to cause yielding such that the 3D growth medium flows to accommodate the group of cells. Movement of a placement tool may be performed manually (e.g. “by hand”) or may performed by a machine or any other suitable mechanism.

In some embodiments, multiple independent groups of cells may be placed within a single volume of a 3D cell growth medium. For example, a volume of 3D cell growth medium may be large enough to accommodate at least 2, at least 5, at least 10, at least 20, at least 50, at least 100, at least 1000, or any other suitable number of independent groups of cells. Alternatively, a volume of 3D cell growth medium may only have one group of cells. Furthermore, it should be understood that a group of cells may comprise any suitable number of cells, and that the cells may of one or more different types.

Depending on the particular embodiment, groups of cells may be placed within a 3D cell growth medium according to any suitable shape, geometry, and/or pattern. For example, independent groups of cells may be deposited as spheroids, and the spheroids may be arranged on a 3D grid, or any other suitable 3D pattern. The independent spheroids may all comprise approximately the same number of cells and be approximately the same size, or alternatively different spheroids may have different numbers of cells and different sizes. In some embodiments, cells may be arranged in shapes such as embryoid or organoid bodies, tubes, cylinders, toroids, hierarchically branched vessel networks, high aspect ratio objects, thin closed shells, or other complex shapes which may correspond to geometries of tissues, vessels or other biological structures.

According to some embodiments, a 3D cell growth medium made from a yield stress material may enable 3D printing of cells to form a desired pattern in three dimensions. For example, a computer-controlled injector tip may trace out a spatial path within a 3D cell growth medium and inject cells at locations along the path to form a desired 3D pattern or shape. Movement of the injector tip through the 3D cell growth medium may impart sufficient mechanical energy to cause yielding in a region around the injector tip to allow the injector tip to easily move through the 3D cell growth medium, and also to accommodate injection of cells. After injection, the 3D cell growth medium may transform back into a solid-like phase to support the printed cells and maintain the printed geometry. However, it should be understood that 3D printing techniques are not required to use a 3D growth medium as described herein.

According to some embodiments, a 3D cell growth medium may be prepared by dispersing hydrogel particles in a liquid cell growth medium. The hydrogel particles may be mixed with the liquid cell growth medium using a centrifugal mixer, a shaker, or any other suitable mixing device. During mixing, the hydrogel particles may swell with the liquid cell growth medium to form a material which is substantially solid when an applied shear stress is below a yield stress, as discussed above. After mixing, entrained air or gas bubbles introduced during the mixing process may be removed via centrifugation, agitation, or any other suitable method to remove bubbles from 3D cell growth medium.

In some embodiments, preparation of a 3D cell growth medium may also involve buffering to adjust the pH of a hydrogel particle and liquid cell growth medium mixture to a desired value. For example, some hydrogel particles may be made from polymers having a predominantly negative charge which may cause a cell growth medium to be overly acidic (have a pH which is below a desired value). The pH of the cell growth medium may be adjusted by adding a strong base to neutralize the acid and raise the pH to reach the desired value. Alternatively, a mixture may have a pH that is higher than a desired value; the pH of such a mixture may be lowered by adding a strong acid. According to some embodiments, the desired pH value may be in the range of about 7.0 to 7.4, or, in some embodiments 7.2 to 7.6, or any other suitable pH value which may, or may not, correspond to in vivo conditions. The pH value, for example may be approximately 7.4. In some embodiments, the pH may be adjusted once the dissolved CO₂ levels are adjusted to a desired value, such as approximately 5%.

Yield stress can be measured by performing a strain rate sweep in which the stress is measured at many constant strain rates. Yield stress can be determined by fitting these data to a classic Herschel-Bulkley model (σ = σ_{y} + kγ̇ⁿ). (b) To determine the elastic and viscous moduli of non-yielded LLS media, frequency sweeps at 1% strain can be performed. The elastic and viscous moduli remain flat and separated over a wide range of frequency, behaving like a Kelvin-Voigt linear solid with damping. Together, these rheological properties demonstrate that a smooth transition between solid and liquid phases occurs with granular microgels, facilitating their use as a 3D support matrix for cell printing, culturing, and assaying.

An example of a hydrogel with which some embodiments may operate is a carbomer polymer, such as Carbopol®. Carbomer polymers may be polyelectrolytic and may comprise deformable microgel particles. Carbomer polymers are particulate, high-molecular-weight crosslinked polymers of acrylic acid with molecular weights of up to 3 -4 billion Daltons. Carbomer polymers may also comprise co-polymers of acrylic acid and other aqueous monomers and polymers such as poly-ethylene-glycol.

While acrylic acid is a common primary monomer used to form polyacrylic acid the term is not limited thereto but includes generally all α-β unsaturated monomers with carboxylic pendant groups or anhydrides of dicarboxylic acids and processing aids as described in U.S. Pat. No. 5,349,030. Other useful carboxyl containing polymers are described in U.S. Pat. No. 3,940,351, directed to polymers of unsaturated carboxylic acid and at least one alkyl acrylic or methacrylic ester where the alkyl group contains 10 to 30 carbon atoms, and U.S. Pat. Nos. 5,034,486; 5,034,487; and 5,034,488; which are directed to maleic anhydride copolymers with vinyl ethers. Other types of such copolymers are described in U.S. Pat. No. 4,062,817 wherein the polymers described in U. S. Pat. No. 3,940,351 contain additionally another alkyl acrylic or methacrylic ester and the alkyl groups contain 1 to 8 carbon atoms. Carboxylic polymers and copolymers such as those of acrylic acid and methacrylic acid also may be cross-linked with polyfunctional materials as divinyl benzene, unsaturated diesters and the like, as is disclosed in U.S. Pat. Nos. 2, 340,110; 2,340,111; and 2,533,635. The disclosures of all of these U.S. Patents are hereby incorporated herein by reference for their discussion of carboxylic polymers and copolymers that, when used in polyacrylic acids, form yield stress materials as otherwise disclosed herein. Specific types of cross-linked polyacrylic acids include carbomer homopolymer, carbomer copolymer and carbomer interpolymer monographs in the U.S. Pharmocopia 23 NR 18, and Carbomer and C10-30 alkylacrylate crosspolymer, acrylates crosspolymers as described in PCPC International Cosmetic Ingredient Dictionary & Handbook, 12th Edition (2008).

Carbomer polymer dispersions are acidic with a pH of approximately 3. When neutralized to a pH of 6-10, the particles swell dramatically. The addition of salts to swelled Carbomer can reduce the particle size and strongly influence their rheological properties. Swelled Carbomers are nearly refractive index matched to solvents like water and ethanol, making them optically clear. The original synthetic powdered Carbomer was trademarked as Carbopol® and commercialized in 1958 by BF Goodrich (now known as Lubrizol), though Carbomers are commercially available in a multitude of different formulations.

Hydrogels may include packed microgels - microscopic gel particles, ~5 µm in diameter, made from crosslinked polymer. The yield stress of Carbopol® is controlled by water content. Carbopol® yield stress can be varied between roughly 1-1000 Pa. Thus, both materials can be tuned to span the stress levels that cells typically generate. As discussed above, while materials may have yield stresses in a range of 1-1000 Pa, in some embodiments it may be advantageous to use yield stress materials having yield stresses in a range of 1-100 Pa or 10-100 Pa. In addition, some such materials may have thixotropic times less than 2.5, less than 1.5 seconds, less than 1 second, or less than 0.5 seconds, and greater than 0.25 seconds or greater than 0.1 seconds, and/or thixotropic indexes less than 7, less than 6.5, or less than 5, and greater than 4, or greater than 2, or greater than 1.

Yield stresses of less than 100 pascals are advantageous as they prevent the formation of unwanted crevasses in the 3D culture medium that detrimentally affects flow of fluid (and nutrient delivery/retrieval) throughout the material. Additionally, yield stresses in this range have advantages for the culture of cells, such as efficient waste retrieval and the ability of cells to expand in their environment without being unnecessarily constrained.

### Bioreactors

Described herein are systems comprising in-situ servo-hydraulic bio-manipulators. Systems as described herein can further comprise one or more bioreactors, for example a perfusion-enabled bioreactor or a perfusion-enabled bioreactor with a passive constant negative pressure device.

Additional aspects of Perfusion-Enabled Bioreactors can be found in PCT/US2019/017316, filed on Feb. 8, 2019, and published as WO 2019/157356 A1 on Aug. 15, 2019, which is incorporated by reference herein in its entirety.

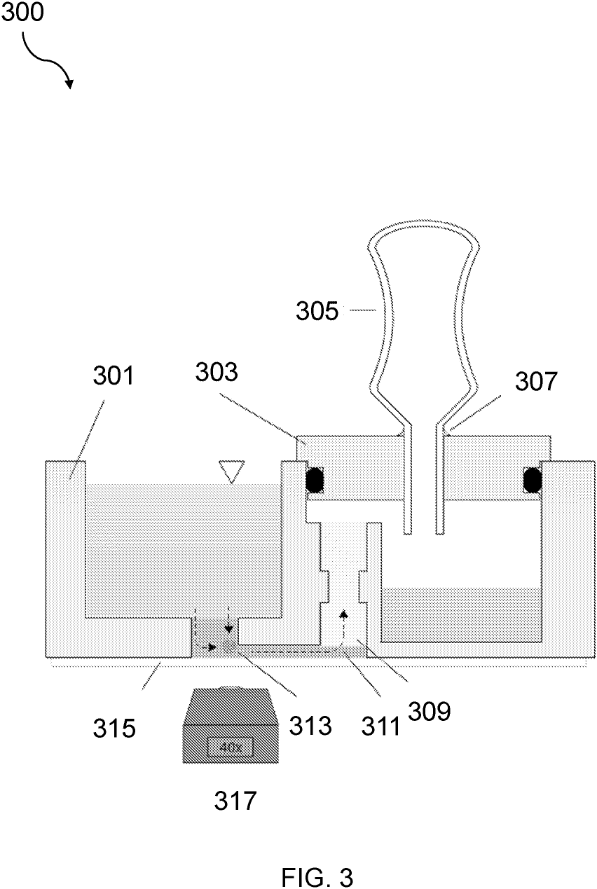

Additional aspects of bioreactors with passive constant negative pressure devices (for example as shown in FIG. 3) can be found in co-pending U.S. Provisional Application number 62/912,396, filed on Oct. 8, 2019 and titled “MICROSCOPY ENABLED PERFUSION BIOREACTOR WELLPLATE WITH PASSIVE NEGATIVE PRESSURE DEVICE”, which is incorporated by reference herein in its entirety.

### Liquid Medium

Liquid medium composition as known in the art, that can be employed in addition to the 3D culture medium as described herein, must be considered from two perspectives: basic nutrients (sugars, amino acids) and growth factors/cytokines. Co-culture of cells often allows reduction or elimination of serum from the medium due to production of regulatory macromolecules by the cells themselves. The ability to supply such macromolecular regulatory factors in a physiological way is a primary reason 3D perfused co-cultures are used. A serum-free medium supplemented with several growth factors suitable for long-term culture of primary differentiated hepatocytes has been tested and found to support co-culture of hepatocytes with endothelial cells. ES cells are routinely maintained in a totipotent state in the presence of leukemia inhibitory factor (LIF), which activates gp130 signaling pathways. Several medium formulations can support differentiation of ES cells, with different cytokine mixes producing distinct patterns of differentiation. Medium replacement rates can be determined by measuring rates of depletion of key sugars and amino acids as well as key growth factors/cytokines. If cell culture medium with sodium bicarbonate is used, the environmental control can be provided by e.g. placing the module with bioreactor/reservoir pairs into a CO₂ incubator.

### Cells

A variety of different cells can be applied to the 3D growth medium of the disclosed systems. In some embodiments, these are normal human cells or human tumor cells. The cells may be a homogeneous suspension or a mixture of cell types. The different cell types may be seeded onto and/or into the medium sequentially, together, or after an initial suspension is allowed to attach and proliferate (for example, endothelial cells, followed by liver cells). Cells can be obtained from cell culture or biopsy. Cells can be of one or more types, either differentiated cells, such as endothelial cells or parenchymal cells, including nerve cells, or undifferentiated cells, such as stem cells or embryonic cells. In one embodiment, the medium is seeded with a mixture of cells including endothelial cells, or with totipotent/pluripotent stem cells which can differentiate into cells including endothelial cells, which will form “blood vessels”, and at least one type of parenchymal cells, such as hepatocytes, pancreatic cells, or other organ cells.

Cells can be cultured initially and then used for screening of compounds for toxicity. Cells can also be used for screening of compounds having a desired effect. For example, endothelial cells can be used to screen compounds which inhibit angiogenesis. Tumor cells can be used to screen compounds for anti-tumor activity. Cells expressing certain ligands or receptors can be used to screen for compounds binding to the ligands or activating the receptors. Stem cells can be seeded, alone or with other types of cells. Cells can be seeded initially, then a second set of cells introduced after the initial bioreactor tissue is established, for example, tumor cells that grow in the environment of liver tissue. The tumor cells can be studied for tumor cell behaviors or molecular events can be visualized during tumor cell growth. Cells can be modified prior to or subsequent to introduction into the apparatus. Cells can be primary tumor cells from patients for diagnostic and prognostic testing. The tumor cells can be assessed for sensitivity to an agent or gene therapy. Tumor cell sensitivity to an agent or gene therapy can be linked to liver metabolism of set agent or gene therapy. Cells can be stem or progenitor cells and the stem or progenitor cells be induced to differentiate by the mature tissue. Mature cells can be induced to replicate by manipulation of the flow rates or medium components in the system.

### Applications

Without intending to be limiting, systems and methods as described herein have many different applications, such as assisting with the identification of markers of disease; assessing efficacy of anti-cancer therapeutics; testing gene therapy vectors; drug development; screening; studies of cells, especially stem cells; studies on biotransformation, clearance, metabolism, and activation of xenobiotics; studies on bioavailability and transport of chemical agents across epithelial layers; studies on bioavailability and transport of biological agents across epithelial layers; studies on transport of biological or chemical agents across the blood-brain barrier; studies on acute basal toxicity of chemical agents; studies on acute local or acute organ-specific toxicity of chemical agents; studies on chronic basal toxicity of chemical agents; studies on chronic local or chronic organ-specific toxicity of chemical agents; studies on teratinogenicity of chemical agents; studies on genotoxicity, carcinogenicity, and mutagenicity of chemical agents; detection of infectious biological agents and biological weapons; detection of harmful chemical agents and chemical weapons; studies on infectious diseases; studies on the efficacy of chemical agents to treat disease; studies on the efficacy of biological agents to treat disease; studies on the optimal dose range of agents to treat disease; prediction of the response of organs in vivo to biological agents; prediction of the pharmacokinetics of chemical or biological agents; prediction of the pharmacodynamics of chemical or biological agents; studies concerning the impact of genetic content on response to agents; filter or porous material below microscale tissue may be chosen or constructed so as bind denatured, single-stranded DNA; studies on gene transcription in response to chemical or biological agents; studies on protein expression in response to chemical or biological agents; studies on changes in metabolism in response to chemical or biological agents; prediction of agent impact through database systems and associated models; prediction of agent impact through expert systems; and prediction of agent impact through structure-based models.

Notably systems and methods as described herein can be utilized for the selection of biological samples, for example for colony selection or the selection of healthy cells or samples from a mixture of tissue (such as a biopsy, for example).

Further applications of the systems and methods as described herein include the manipulation of non-living in organic materials, such as signaling markers and/or dyes. For example, the construction and maintenance of perfusion experiments outside the realm of manipulation of organic tissue is an application of the systems and methods as described herein. In embodiments, inorganic (i.e. non-living) bio-fluorescent beads can be located adjacent to live tissue to signal local protein and antigen concentrations as well as flow conditions. Signaling markers including Phenol red and Bromophenol blue can be printed and/or manipulated near relevant structures (such as groupings of living cells) to indicate or otherwise report physiological conditions such as local pH concentrations in various . Inorganic scaffolding for tissue growth can be printed in perfusing environments, and reference objects can be placed for image analysis.

A number of embodiments of the present disclosure have been described. Nevertheless, it will be understood that various modifications may be made without departing from the spirit and scope of the present disclosure. Accordingly, other embodiments are within the scope of the following claims.

In addition, the following embodiments and features can be incorporated into one or more aspects or embodiments as provided herein. The following are provided to illustrate additional features that can be incorporated together with embodiments provided above and herein as well as with one or more of each other. The present disclosure is not limited to each feature independently, rather various combinations of one or more of these features with one or more of the features disclosed above and herein in contemplated.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

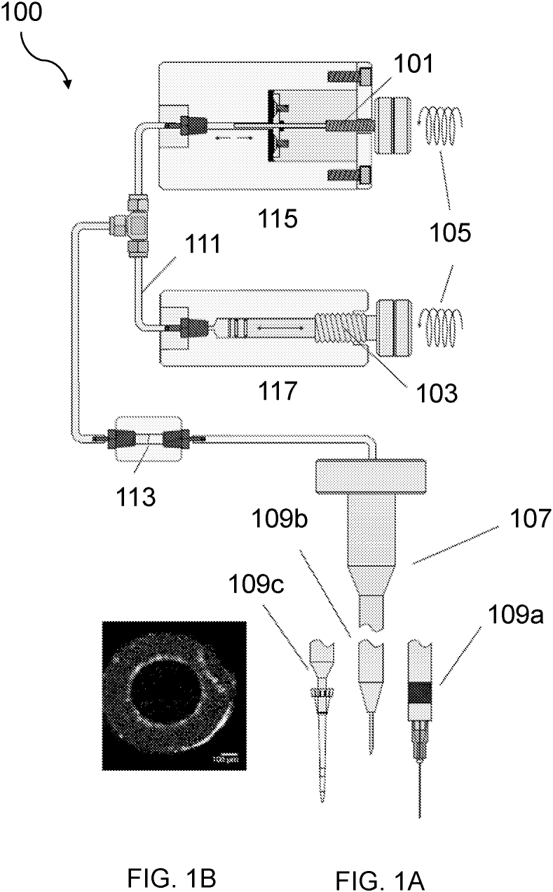

Retrieval of spheroids exhibiting anomalous or desirable behavior became of interest during in situ imaging assays. FIG. 1A is a schematic showing an embodiment of an in-situ servo-hydraulic bio-manipulator 100 as described herein.

To circumvent limitations due to printing dispersions and the likelihood of cell damage during bulk retrieval, a mechanism 115 was constructed which could displace volumes on the order of 0.05 µL as shown in FIG. 1 (with a threaded rod 101, for example a 3/16-1000 UNUF threaded rod for micro-displacements). A macro-displacement hydraulic controller 117 is also shown that can displace volumes on the order of 1ml/rotation upon rotation of the threaded shaft 103 (for example a ½-13 UNC threaded shaft). Rotating the dial of either controller 105 results in the axial translation of a precision ground shaft through the thread helix 101 or 103, thereby displacing a corresponding fluid volume. The system is filled with Novec 7500 fluid 111 to prevent the formation of excess bubbles and meets an immiscible barrier of PBS 113 which imparts biocompatibility.

A head assembly 107 is shown that can fix to the optical turret of a microscope, for example a Nikon A1R confocal microscope. Embodiments of displacement tips 109a (sealed adapter compatible with Luer-Lock syringes), 109b (glass capillary for manipulation of ultra-low volumes), and 109c (tapered nozzle for mounting sterilized micropipette tips, for example 10, 20, 200 microliter tips. FIG. 1B illustrates the visual feedback of tip location through DAPI fluorescence and bright-field imaging that provides an effective means of positioning the tip over select biomaterials.

### Example 2

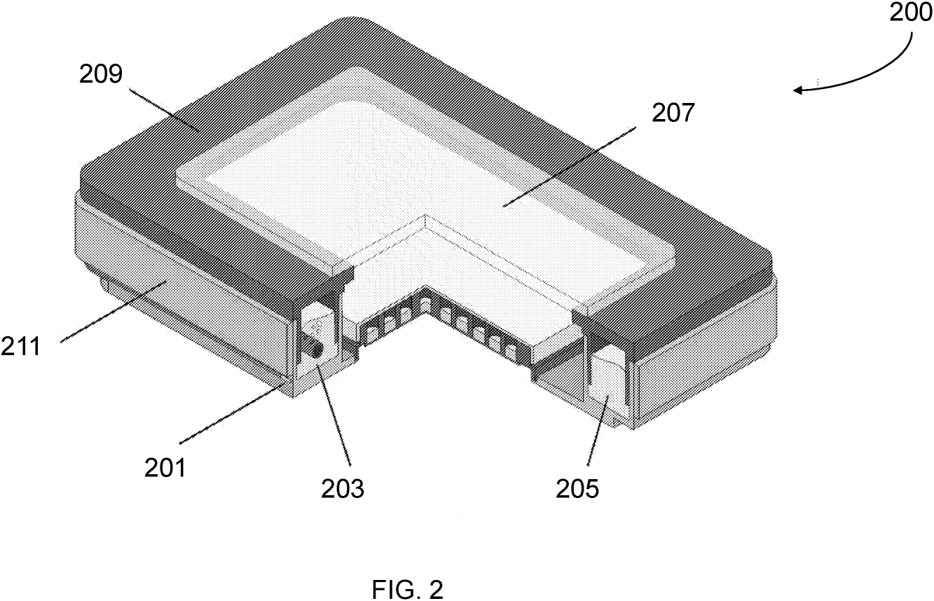

Development of a series of incubators capable of maintaining ideal environmental conditions and interfacing with the Nikon A1R HD25 confocal microscope was important for preserving cell viability and validating experimental results. FIG. 2 is an embodiment of a confocal compatible incubator 200 which can be used as part of a bio-manipulation system in conjunction with in-situ servo-hydraulic bio-manipulators as described herein.

FIG. 2 provides a sectional view of an embodiment of a 96-well plate revision used during staining and blocking solution procedures. Incubators were also constructed for imaging perfusion plates and culture dishes for additional versatility.

Aspects of the confocal compatible incubator 200 include a thermocouple (for example a type K thermocouple); a perforated bubbling line 203 that can produce humidified gasses for the imaging chamber; a fluid jacket 205 (for example a water jacket) that can reduce the risk of thermal fluctuations and increases thermal mass of the system; a transparent lid 207 (for example an acrylic lid) for observation during incubation; a heat insulation lid 209 (for example a Delrin lid); and a heating element 211 (for example a 10W/in² adhered heating element).

### Example 3

Performing kinetic studies requires in vitro imaging capabilities incompatible with end-point assays. A disposable miniaturized well plate 300 was previously constructed to circumvent these limitations (FIG. 3) that can be used in conjunction with systems and methods as described herein. FIG. 3 is an embodiment of a perfusion-enabled bioreactor which can be used as part of a bio-manipulation system in conjunction with in-situ servo-hydraulic bio-manipulators as described herein. The embodiment of FIG. 3 can be utilized along with negative pressure vessels to enable perfusion of fluids (delivery of nutrients and removal of cellular waste) throughout the bioreactor.

The embodiment of the imaging perfusion plate revision illustrated in FIG. 3 identifies several key improvements over previous iterations including an expanded initial volume for longer perfusion durations as well as simplified construction (for example using acrylic construction 301 and 303 to monitor perfusion). A transfer pipet 305 was introduced in this design which when compressed can induce a ~ 5 kPa pressure differential within the system along with a biocompatible adhesive seal 307. The pHEMA barrier 309 prevents LLS 311 drift between wells and the o-ring seal effectively protects from leakage (dark circle lateral to each side of the compressed transfer pipette nozzle). A glass coverslip 315 (for example about ~0.2 mm thick) is adhered to the anterior of the plate using biocompatible optical glue providing adequate space for higher magnification objectives 317 than previously capable.

### Example 4

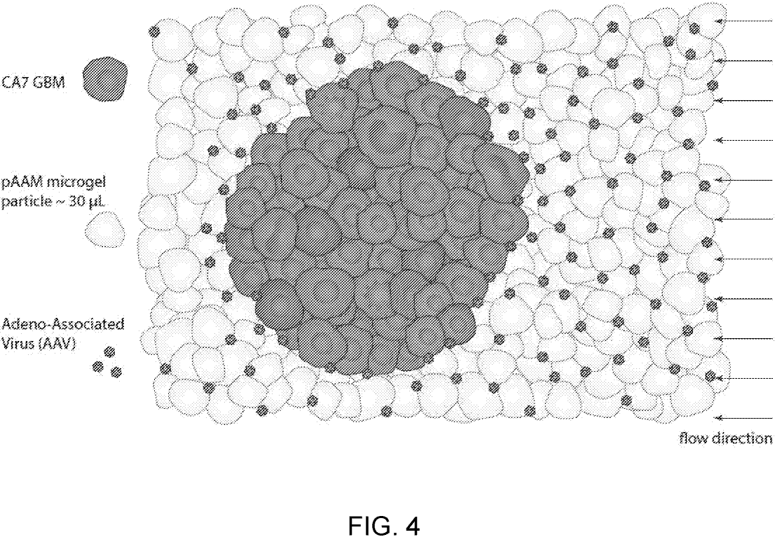

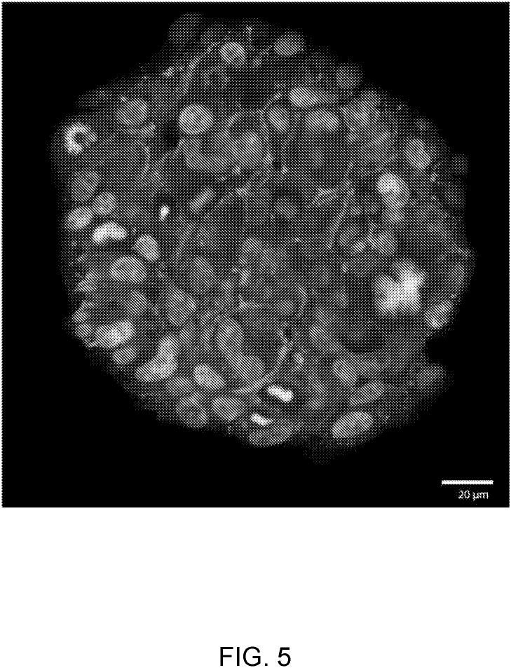

FIGS. 4 and 5 illustrate applications of systems and methods as described herein.

An extended duration kinetics study required selection and retrieval of spheroids in situ, long-term imaging periods under incubation to preserve cell viability, and perfusion to remove waste and flow.

Adeno-Associated Viruses (AAVs) across 3D biofabricated tumoroids glioblastoma spheroids (FIG. 4). These AAVs carried an RNA package which coded for the production of Green Fluorescent Protein (GFP) in the cytoplasm. After 6 days of perfusion a fixation protocol was successfully performed within the imaging perfusion plate and the spheroids were removed using the micromanipulator and stained. Cell nuclei were stained DAPI with intercellular junctions stained TRITC (FIG. 5).

### Example 5

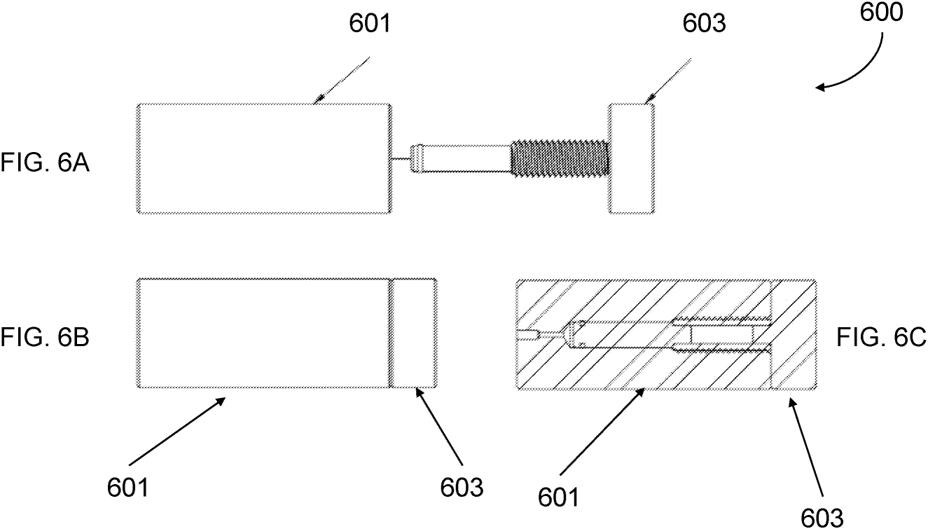

FIGS. 6-8 illustrate embodiments of components of in-situ servo-hydraulic bio-manipulators as described herein.

FIGS. 6A-6C show an embodiment of a macro-displacement hydraulic controller 600 according to the present disclosure. FIG. 6A shows an exploded unassembled view; FIG. 6B is an assembled view; and FIG. 6C is a cross-sectional assembled view. As shown in FIGS. 6A-6C, a macro control dial 603 (for example a 130 uL controller dial, SMEL - 130 uL - 002) and a macro control housing 601 (for example a 130 uL housing, SMEL - 130 uL - 001) .

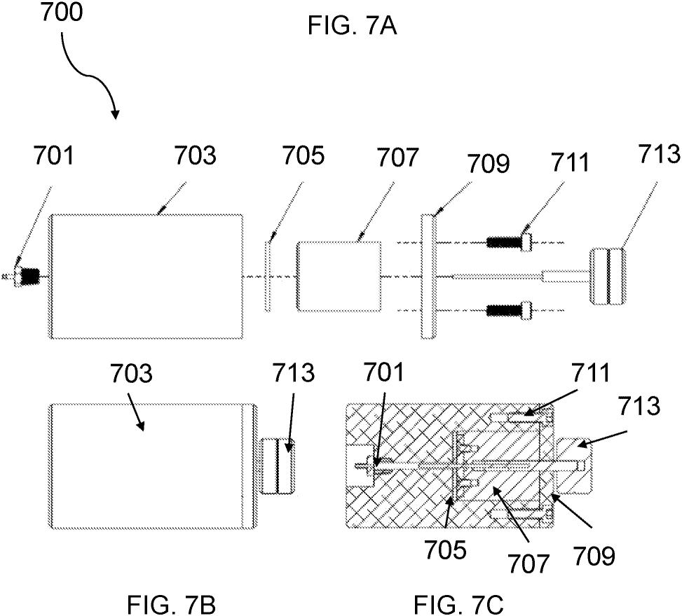

FIGS. 7A-7C shows an embodiment of a micro-displacement hydraulic controller 700 according to the present disclosure. FIG. 7A shows an exploded unassembled view; FIG. 7B is an assembled view; and FIG. 7C is a cross-sectional assembled view. As shown in FIGS. 7A-7C, a micro controller dial 713 (for example a 0.5 microliter control dial) is threaded through a controller housing cap 709 and internal sealing subassembly 707 and internal gasket 705 into the controller housing 703 (affixed by screws 711) and fitted with a fitting 701 (for example a 1/16 NPT fitting).

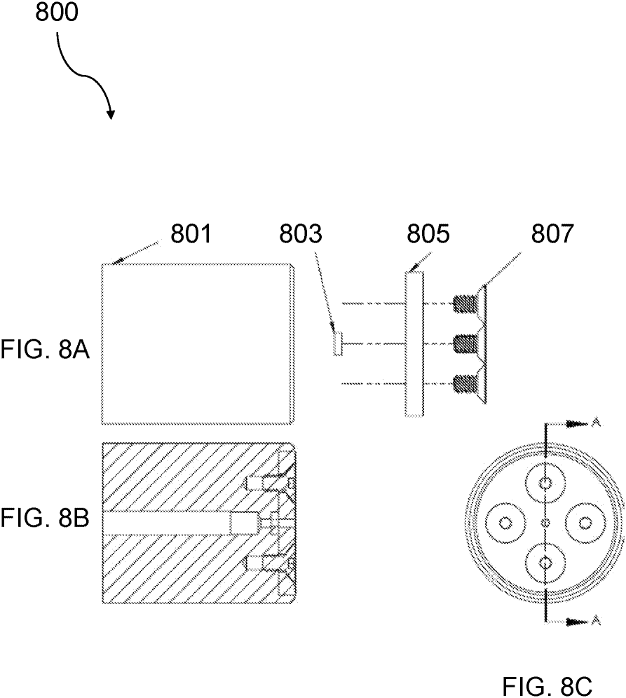

FIGS. 8A-8C shown an embodiment of an internal sealing assembly according to the present disclosure. FIG. 8A shows an exploded unassembled view; FIG. 8B is a cross-sectional assembled view through the plane “A” of FIG. 8C; and 8C is an end view. As shown in FIGS. 8A-8C, an internal sealing face plate 805 is secured to the internal sealing assembly housing 801 with screws 807 (and an o-ring 803 between the internal sealing face plate 805 and internal sealing assembly housing 801).

### Example 6

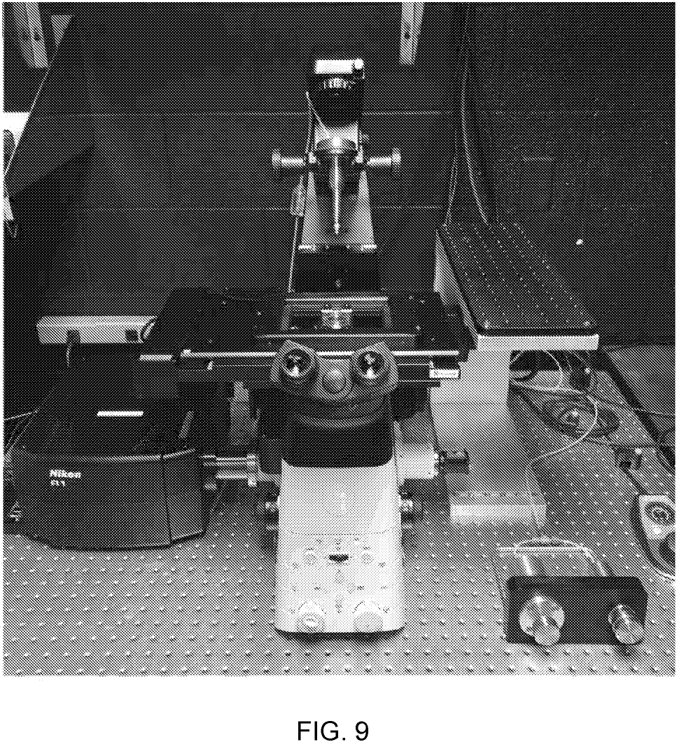

FIGS. 9-12 are photographs that depict aspects of a reduced-to-practice embodiment of the present disclosure.

FIG. 9 is a photograph showing a reduced-to-practice embodiment of a bio-manipulator coupled a confocal microscope. Bio-manipulator controls and assemblies shown in a standard working layout. The dial assemblies are located near the operator for making small volume displacements. During a print, the turret head assembly is tilted over the culture infrastructure and the needle tip is lowered into the sample using the geared travel native to the confocal microscope. The material manipulation is completed using either brightfield or fluorescent imaging, after which the needle is raised out of the sample and tilted away before removal.

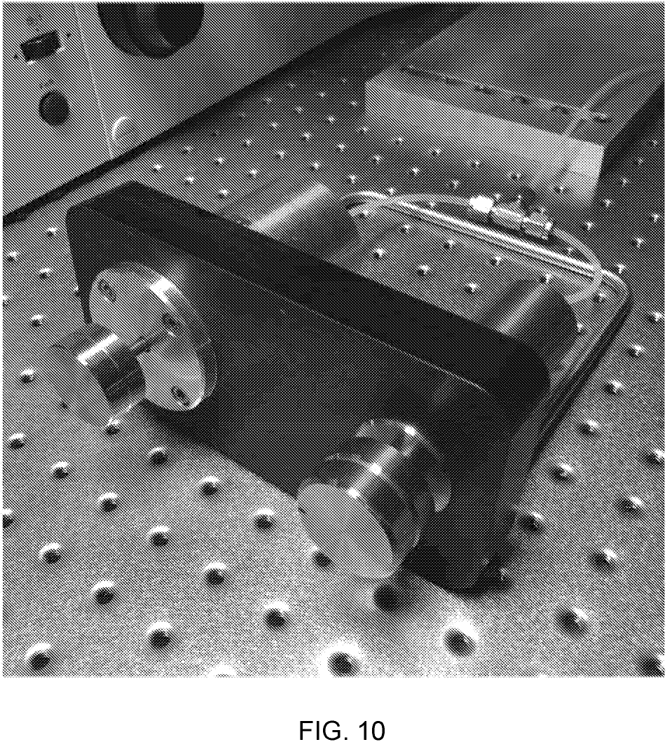

FIG. 10 is a photograph showing a reduced-to-practice embodiment of a bio-manipulator controller assembly of a bio-manipulator as described herein. The controller assembly shown with both the coarse (150 uL/revolution) and fine (0.5 uL/revolution) dial assemblies integrated into the Delrin/stainless steel frame. Rotation of either dial produces axial travel along its respective internal thread helix, which in turn displaces the Novec 7500 engineering fluid. The coarse dial incorporates a 1 /2-13 UNC thread, while the fine dial incorporates a 3/16-100 UNEF thread. Both dials can be operated independently to accomplish certain tasks, with the fine dial being used for manipulating biomaterials while the coarse dial is used for both clearing needle tips and exchanging turret assemblies. Dial assemblies are connected by a T-fitting which is then connected to the transparent junction box through flexible tubing.

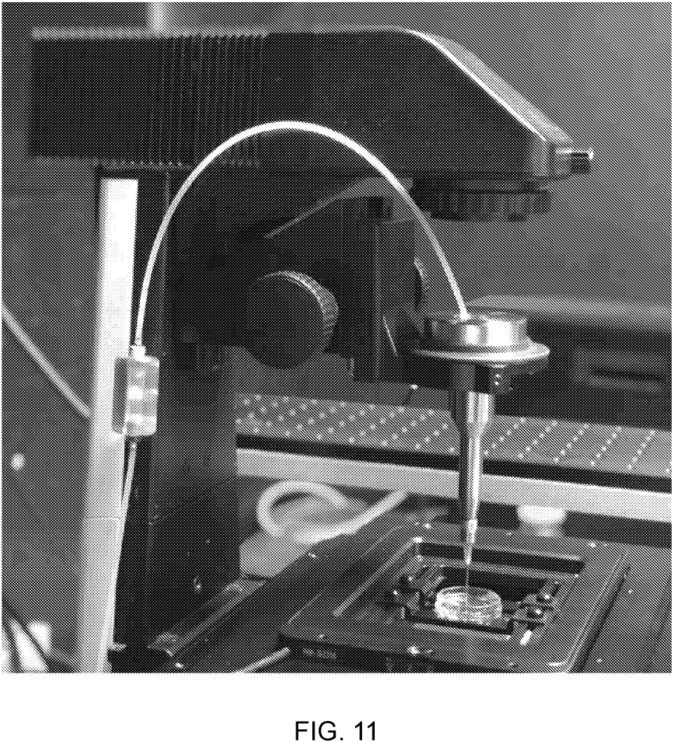

FIG. 11 is a photograph showing a reduced-to-practice embodiment of a bio-turret head assembly for a bio-manipulator as described herein. Turret head assembly shown with the syringe needle attachment variant. The acrylic junction box (shown affixed to the column of the confocal microscope) provides visibility to the Novec 7500 engineering fluid and Phosphate Buffered Saline (PBS) immiscible layer. The location of the transparent junction box can be adjusted to reduce the height of the column of liquid acting at the printing interface to prevent unintended flow/suction. Different printing heads can be attached to the junction box through removal of the flexible tubing with care not to introduce any unintended cavities or bubbles.

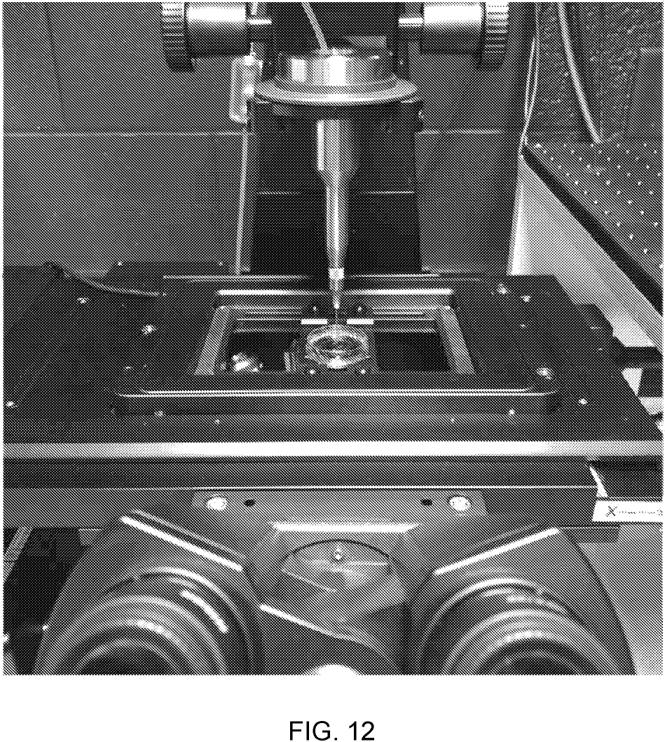

FIG. 12 is a photograph showing a reduced-to-practice embodiment of a bio-turret head assembly for a bio-manipulator as described herein. Illustration of a typical print/extraction setup using the syringe needle variant of the turret head assembly. The turret head assembly mounts to the turret of the confocal microscope and is aligned along the optical axis simplifying the process of locating the needle tip relative to the materials being manipulated. In addition, the vertical orientation of the needle and turret head assembly improves upon the versatility of the system when working with culture plates and infrastructure with relatively tall cavities. Traditional micro-manipulators are oriented at an angle mounted away from the optical axis, reducing clearance. The stage of the confocal microscope translates on the x-y coordinate plane, while the turret is translated along the z-axis using a geared-head. When not in use, the turret head assembly is tilted away from the print-site and disconnected.

Unless defined otherwise, all technical and scientific terms used herein have the same meanings as commonly understood by one of skill in the art to which the disclosed present disclosure belongs. Publications cited herein and the materials for which they are cited are specifically incorporated by reference.

Those skilled in the art will recognize, or be able to ascertain using no more than routine experimentation, many equivalents to the specific embodiments of the present disclosure described herein. Such equivalents are intended to be encompassed by the following claims.

Nevertheless, it will be understood that various modifications may be made without departing from the spirit and scope of the present disclosure. Accordingly, other embodiments are within the scope of the following claims.

What is claimed is:

1. An in-situ servo-hydraulic bio-manipulator, comprising
a micro-displacement hydraulic controller;
a macro-displacement hydraulic controller;
a junction box, wherein a portion of the junction box is optically transparent;
an extrusion head in fluidic communication with the junction box, micro-displacement controller, and macro-hydraulic controller; and
an adapter configured to mechanically couple the extrusion head to the optical axis of a microscope.

2. The in-situ servo-hydraulic bio-manipulator of claim 1, wherein the extrusion head further comprises an adapter configured to receive interchangeable tips.

3. The in-situ servo-hydraulic bio-manipulator of claim 2, wherein the adapter configured to receive interchangeable tips is a tapered nozzle configured to receive micropipette tips, a tapered nozzle with an orifice configured to receive a glass capillary, or a sealed adapter configured to receive a luer-lock syringe needle.

4. The in-situ servo-hydraulic bio-manipulator of claim 1, wherein the micro-displacement hydraulic controller comprises an internal sealing assembly.

5. The in-situ servo-hydraulic bio-manipulator of claim 1, wherein the micro-displacement hydraulic controller and macro-displacement hydraulic controller are in fluidic communication with the junction box through tubing filled with a first fluid, and
the extrusion head and junction box are in fluidic communication though tubing filled with a second fluid, wherein the second fluid is different than the first fluid.

6. The in-situ servo-hydraulic bio-manipulator of claim 5, wherein the first fluid is a non-biocompatible fluid.

7. The in-situ servo-hydraulic bio-manipulator of claim 5, wherein the first fluid is Novec 7500.

8. The in-situ servo-hydraulic bio-manipulator of claim 5, wherein the second fluid forms an immiscible layer with the first fluid in the junction box.

9. The in-situ servo-hydraulic bio-manipulator of claim 5, wherein the second fluid is a bio-compatible fluid.

10. The in-situ servo-hydraulic bio-manipulator of claim 5, wherein the second fluid is phosphate-buffered saline (PBS).

11. The in-situ servo-hydraulic bio-manipulator of claim 1, wherein the micro-displacement hydraulic controller and macro-displacement hydraulic controller each comprise a threaded shaft, the threaded shaft of the macro-displacement hydraulic controller being larger in diameter than the threaded shaft of the micro-displacement hydraulic controller.

12. The in-situ servo-hydraulic bio-manipulator of claim 11, wherein the threaded shaft of the of the macro-displacement hydraulic controller is a ½-13 UNC threaded shaft or a 3/16-100 UNUF threaded rod.

13. (canceled)

14. The in-situ servo-hydraulic bio-manipulator of claim 1, wherein the micro-displacement hydraulic controller and macro-displacement hydraulic controller each comprise a dial capable of being operated independently of the other.

15. A bio-manipulation system, comprising:
an in-situ servo-hydraulic bio-manipulator of claim 1; and
a bioreactor.

16. The bio-manipulation system of claim 15, wherein the bioreactor is a perfusion-enabled bioreactor.

17. The bio-manipulation system of claim 16, wherein the perfusion-enable bioreactor comprises a passive negative constant pressure device.

18. The bio-manipulation system of claim 15, further comprising a 3D cell growth media in the bioreactor.

19. The bio-manipulation system of claim 18, wherein the 3D cell growth media is a Herschel-Bulkley fluid having a yield stress of less 100 pascals.

20. A method of using an in-situ servo-hydraulic bio-manipulator, comprising:
providing an in-situ servo-hydraulic bio-manipulator of claim 1;
providing one or more mammalian cells; and
translating the position of the one or more mammalian cells by operating the micro-displacement hydraulic controller, macro-displacement hydraulic controller, or both.

21. A method of using an in-situ servo-hydraulic bio-manipulator, comprising:
providing an in-situ servo-hydraulic bio-manipulator of claim 1;
providing one or more inorganic signaling markers; and
translating the position of the one or more inorganic signaling markers by operating the micro-displacement hydraulic controller, macro-displacement hydraulic controller, or both.
